(19) United States
(12) Patent Application Publication
(10) Pub. No.: US 2025/0032003 A1
(43) Pub. Date: Jan. 30, 2025

(54) JAW MOVEMENT TRACKING SYSTEM AND METHOD

(71) Applicant: JAWSAVER B.V., UTRECHT (NL)

(72) Inventors: Gabriel Enrique SAENZ, UTRECHT (NL); Selina TIRTAJANA, UTRECHT (NL); David Benjamin JAROCH, ARVADA, CO (US); Joost PLATTEL, UTRECHT (NL)

(21) Appl. No.: 18/715,148

(22) PCT Filed: Nov. 29, 2022
(86) PCT No.: PCT/EP2022/083568

§ 371 Date: May 31, 2024

(30) Foreign Application Priority Data
Dec. 1, 2021 (NL) 2029986
Jul. 12, 2022 (NL) 2032452

(51) Int. Cl. A61B 5/11; A61B 5/00; G16H 40/63

(52) U.S. Cl. CPC A61B 5/1126; A61B 5/6817; A61B 5/7246; A61B 5/7264; A61B 5/7282; A61B 5/7405; A61B 5/7455; G16H 40/63; A61B 5/4557; A61B 5/4812; A61B 5/4818; A61B 2560/0462; A61B 2562/0223; A61B 2562/164

(57) ABSTRACT

Known jaw movement tracking systems generally allow for a limited tracking of the jaw movement. e.g. they are only able to track the jaw movement in two dimensions or track muscle activations without specifying direction of movements, and are thus unable to fully track the complex jaw movements. The reduced tracking ability of known devices reduces the ability to discriminate between different jaw movements based on the tracked movement. A need exists for a jaw movement tracking system that allows jaw movement tracking for discriminating between different jaw movements. The invention relates to a jaw movement tracking system. invention further relates to a method for use of the jaw movement tracking system.

### CROSS-REFERENCE TO RELATED APPLICATIONS

This application is the National Stage of International Application No. PCT/EP2022/083567, filed Nov. 29, 2022, which claims priority to NL 2029986, filed Dec. 1, 2021, all of which are entirely incorporated herein by reference.

### FIELD OF THE INVENTION

The invention relates to a jaw movement tracking system. The invention further relates to a method for use of the jaw movement tracking system.

### BACKGROUND OF THE INVENTION

The jaw is attached to the skull of a person via the temporomandibular joint (TMJ), which is one of the most complex joints in the human body. Unlike a hinge joint, such as the elbow joint, that has limited two-dimensional movements, the TMJ allows the jaw to slide over the surface of the skull bone while rotating, allowing for complex jaw movements.

Tracking jaw movement may be used in various applications. For example, tracking jaw movement may be used to:
detect bruxism;
determine a sleep stage;
detect signals of sleep-breathing disorders, e.g. apnea, or hypopnea;
monitor eating behaviour;
detect signals of temporomandibular joint disorder;
determine a user sleep position, e.g. for sleep tracking, positional sleep apnea therapy;
hands-free control of devices via jaw gesture detection
provide a biometric authentication via silent speech recognition; and
detect other behaviour involving jaw movements, such as smoking.

Known jaw movement tracking systems generally allow for a limited tracking of the jaw movement, e.g. they are only able to track the jaw movement in two dimensions or track muscle activations without specifying direction of movements, and are thus unable to fully track the complex jaw movements. The reduced tracking ability of known devices reduces the ability to discriminate between different jaw movements based on the tracked movement. A need exists for a jaw movement tracking system that allows jaw movement tracking for discriminating between different jaw movements.

### OBJECT OF THE INVENTION

It is an aim of the invention to provide an improved jaw movement tracking system. It is a further aim of the invention to provide a jaw movement tracking system that allows discriminating between different jaw movements, e.g. allowing to discriminate between bruxism and an eating behaviour or between a sleep stage and smoking.

### SUMMARY OF THE INVENTION

The aim of the invention is achieved by the jaw movement tracking system of claim 1.

The jaw movement tracking system comprises a first in-ear device, that can be placed in the left or right ear of a user, for measuring a movement of the first TMJ, e.g. of the left or right TMJ of a user. The use of an in-ear device is advantageous over other devices in that an in-ear device is less intrusive and may be worn throughout the day and night without being obvious to an observer, and without being uncomfortable.

The first in-ear device comprises a first in-ear portion configured to be placed in the first ear canal, for example, a left ear canal of a user making the first in-ear portion a left in-ear portion, wherein the first in-ear portion comprises a first distal portion made from a shape conforming material that conforms to the shape of the first ear canal when the first in-ear portion is placed in the first ear canal. The shape of the first ear canal of a person is dependent on the position of the jaw because the ear canal is adjacent to the TMJ such that the jaw and/or the TMJ press against the ear canal. By making the first distal portion of a shape conforming material, the first distal portion may conform its shape to the shape of the first ear canal. The shape of the first distal portion, when placed in the first ear canal, is thus indicative of the jaw position and jaw movement of the person. For example, the first in-ear device comprises an earbud constructed and configured to be positioned in a wearer's ear canal and having a distal portion situated farther in the ear canal than a proximal portion. The proximal portion which is designed to fit securely in the wearer's ear to minimize movement while the distal portion is made from a shape conforming material that conforms to the wearer's ear canal when the earbud is placed in the ear, and can move/change its shape along with the deformation of the ear canal that occurs during various activities where the TMJ is involved.

The first in-ear device further comprises a first magnet. For example, the first magnet is provided in the first distal portion adjacent to the first temporomandibular joint, e.g. the TMJ adjacent to the first ear canal, when the first in-ear portion is placed in the first ear canal. Thus in this example the first magnet is provided on the shape conforming material of the first distal portion. This allows the position of the magnet, when the in-ear device is placed in the first ear canal, relative to another portion, e.g. a proximal portion, of the in-ear device to depend on the shape of the ear canal and thus on the jaw movement and position. The first magnet may be embodied as a single magnet, an array of magnets, an electromagnet, a strip of magnets or a piece of material with magnetic properties. The first magnet may be any object, such as a ferromagnet, an electromagnet, a suitably dense metal, creating a magnetic field detectable by the first magnetic sensor.

The first in-ear device further comprises a first magnetic sensor for measuring a movement of the first magnet relative to the first magnetic sensor by detecting a change in magnetic flux density generated by the movement of the first magnet. For example, if the first magnet is provided in the distal portion, as the first magnet moves under influence of a change of shape of the first ear canal, the relative position of the first magnet to the magnetic sensor changes. This allows the first magnetic sensor to detect a change in the magnetic field generated by the magnet. This change in the magnetic field is indicative of the relative position of the first magnet to the first magnetic sensor and of the jaw movement and position. This allows the first magnetic sensor to measure a signal indicative of the jaw position and jaw movement. For example, the magnetic sensor may measure flux density changes along an X, Y and Z direction and the magnetic sensor and/or the processor may be configured to monitor a subset, e.g. only the X, and Y directions, of these directions.

The in-ear device allows either the first magnet or the first magnetic sensor to be provided in the distal portion, e.g. wherein the first magnet or the first magnetic sensor is provided adjacent to the temporomandibular joint when the first in-ear portion is placed in the first ear canal, and e.g. wherein the other one of the first magnet and the first magnetic sensor is provided in a proximal portion or a support. Thus under movement of the first TMJ either the first magnet or the first magnetic sensor may be displaced causing a relative movement of the first magnet relative to the first magnetic sensor.

The jaw movement tracking system further comprises a processor which is connected to the first magnetic sensor for receiving the measured movement of the first magnet relative to the first magnetic sensor. The processor may be provided in the first in-ear device, e.g. in an out-of ear portion, or the processor may be provided separately from the first in-ear device, e.g. implemented in a carriable device, e.g. such as a watch.

The processor is configured to:
determine a first jaw movement based on the measured movement of the first magnet relative to the first magnetic sensor, e.g. wherein the first jaw movement is determined based on a model, e.g. an empirical model, relating movement of the magnet to jaw movement;
determine one or more first characteristics of the first jaw movement, e.g. velocity of the first jaw movement, direction of the first jaw movement, amplitude of the first jaw movement, and frequency of the first jaw movement, based on the determined first jaw movement;
compare the determined first characteristics to one or more indicative characteristics that are indicative of a predetermined event to determine an occurrence of the event, e.g. wherein an occurrence of an event is determined when the determined first characteristics have a predetermined overlap with the indicative characteristics; and
output an event signal when the processor determines the occurrence of the event.

The processor is configured to determine a first jaw movement based on the measured movement of the first magnet relative to the first magnetic sensor, relating movement of the first magnet as measured by the change in flux density to jaw movement. The processor may use an empirical model, a regression model, or any other type of suitable model to relate the jaw movement to the measured relative movement. As explained above the movement of the first magnet relative to the first magnetic sensor is indicative of the jaw movement because the jaw movement influences the shape of the first ear canal which influences the position of the first magnet relative to the first magnetic sensor.

The processor is configured to determine one or more first characteristics of the first jaw movement. For example, the processor may be configured to determine a velocity of the first jaw movement, a direction of motion of the first jaw, an amplitude of the first jaw movement and a frequency of the first jaw movement from the determined first jaw movement, e.g. which may vary over time. Thus the determined first jaw movement results in one or more first characteristics that are characteristic of the jaw movement.

The processor is further configured to compare the determined one or more first characteristics to one or more indicative characteristics that are indicative of a predetermined event, e.g. bruxism, sleep stage, etc, to determine an occurrence of the event. For example, wherein the occurrence of the event is determined when the determined first characteristics have a predetermined, e.g. sufficient, overlap with the indicative characteristics. It was found that specific behaviours of a person give rise to specific characteristics of the jaw movement. By comparing the determined first characteristics to the one or more indicative characteristics, that may be chosen depending on the event to be determined, the processor may determine the occurrence of the event. For example, the processor may be configured to determine occurrence of a specific event by comparing the first characteristics to different sets of indicative characteristics, e.g. wherein each set is indicative of a different event, the processor may then determine what the specific event is by looking at which set of indicative characteristics has the most overlap with the measured first characteristics.

The processor is further configured to output an event signal, e.g. in the form of a signal displayed on a display, when the processor determines the occurrence of the event. The event signal may comprise information on the event, e.g. type of event, intensity of event, duration of event, times the event has occurred, etc.

The aim of the invention is achieved by the jaw movement tracking system because the movement of the first magnet relative to the first magnetic sensor is directly related to the movement of the TMJ. Thus a specific movement of the TMJ is converted to a specific movement of the first magnet relative to the first magnetic sensor, which may be measured by the first magnetic sensor. This allows discriminating, e.g. improved discriminating, between different jaw movements and allows the system to discriminate between various behaviours such as bruxism, an eating disorder, a sleep stage, etc.

In embodiments, the in-ear device further comprises:
a second in-ear device comprising:
   a second in-ear portion configured to be placed in a second ear canal, wherein the second in-ear portion comprises a second distal portion made from a shape conforming material that conforms to a shape of the second ear canal when the second in-ear portion is placed in the second ear canal;
   a second magnet;
   a second magnetic sensor for measuring movement of the second magnet relative to the second magnetic sensor by detecting a change in magnetic flux density generated by the movement of the second magnet,
      wherein either the second magnet is provided in the second distal portion, e.g. wherein the second magnet is provided adjacent to the second temporomandibular joint when the second in-ear portion is placed in the second ear canal or wherein the magnetic sensor is provided in the distal portion,
      wherein the processor is connected to the second magnetic sensor for receiving the second measured movement,
      and wherein the processor is further configured to:
determine a second jaw movement based on the second measured movement of the second magnet relative to the second magnetic sensor;
determine one or more second characteristics of the second jaw movement, e.g. velocity of the second jaw movement, direction of the second jaw movement, amplitude of the second jaw movement, and frequency of the second jaw movement, based on the second jaw movement;
compare the determined second characteristics to one or more indicative characteristics that are indicative of a predetermined event to determine an occurrence of the event; and
output the event signal when the processor determines the occurrence of the event.

It was found that, depending on the person, one or the other ear canal is more suitable for tracking jaw movement. For example, when a person is in the bed and lying on their right side, the left ear canal may be more suitable for tracking jaw movements. In another example, the person may be moving between lying on their right side and their left side. In another example, a person's TMJ is such that shape changes in one ear canal are more pronounced than in the other ear canal. Thus, the system benefits from placing a second in-ear device in the second ear canal, allowing to measure changes in shape of both ear canals, and thus to be able to track the first jaw movement based on the first ear canal, and the second jaw movement based on the second ear canal. Both jaw movements may then result in one or more characteristics, e.g. first and second characteristics, which are compared to the indicative characteristics for determining occurrence of an event. This allows the system to better determine the occurrence of an event. The second magnet may be any object, such as a ferromagnet, an electromagnet, a suitably dense metal, creating a magnetic field detectable by the second magnetic sensor.

In further embodiments, the processor is further configured to:
compare the first characteristics to the second characteristics, e.g. by comparing which of the determined characteristics is closer to the one or more indicative characteristics; and
determine a relative relevance of the determined characteristics.

In these embodiments, the processor is configured to determine a relative relevance of the determined characteristics, e.g. determined via a first in-ear device placed in a left ear canal and a second in-ear device placed in a right ear canal, which may be used to determine an occurrence of an event based on a weighted comparison of the first and second characteristics with the indicative characteristics. For example, if the first characteristics are found to be more relevant than the second characteristics, e.g. because the signal of the first characteristics is stronger, than the occurrence of the event may be determined based on the first characteristics alone.

In embodiments, the predetermined event is one of bruxism, sleep stage, sleep-breathing disorders, e.g. apnoea, or hypopnea, eating, abnormal temporomandibular joint movement, provide a biometric authentication via silent speech recognition, jaw movement for providing hands-free jaw gesture control for a device and smoking. For example, the predetermined event may be one or more of the behaviours of the embodiment. For example, the jaw movement tracking system may be used to determine an identity of a person based on a biometric authentication via silent speech recognition. For example, the predetermined event may be a predetermined jaw movement correlated with a command for a device, such that performance of the jaw movement is detected as the predetermined event and the command is sent to the device.

In embodiments, the first magnetic sensor and/or the second magnetic sensor is a hall effect sensor. In other embodiments the first magnetic sensor and/or the second magnetic sensor may be magnetoresistive elements, Hall effect sensors, and/or multipolar magnetic sensors. The Hall effect sensor is configured to measure voltage output that is proportional to the magnetic flux density sensed along x, y and z axes, which is inversely proportional to the distance between the sensor and the first and/or second magnet. For example, the x-movement corresponds to a jaw protrusion movement, the y-movement corresponds to a jaw opening and closing movement and the z-movement corresponds to a lateral jaw movement. Advantageously, the Hall effect sensor is generally small enough, and requires sufficient little power, which allows it to be integrated into smaller in-ear devices which may be more comfortable to wear and may reduce stigma of using such a device.

In embodiments, the first magnetic sensor and/or the second magnetic sensor are configured to determine a relative rotational movement of the first and/or second magnet, and/or a relative linear movement of the first and/or second magnet. For example, the first magnet and/or the second magnet may be a longitudinal magnet, having one axis relatively longer than the other two axis, resulting in a measurable change in flux when the magnet is rotated relative to the first magnetic sensor or the second magnetic sensor.

In embodiments, the tracking system further comprises a feedback device connected to the processor, wherein the feedback device is configured to provide a feedback stimulus when the feedback device receives the event signal from the processor. The processor may be provided on the feedback device. These embodiments are advantageous, because the feedback device may be used to provide a biofeedback stimulus to the user when the user is performing an unwanted behaviour, such as bruxism. This allows the user to become aware of the behaviour and to stop the behaviour. This may result in the user learning not to perform the behaviour. The feedback device may also be multiple feedback devices or a single feedback device capable of providing several distinct feedback stimuli. The feedback may be a strong feedback stimulus that consciously alerts the wearer, and the feedback may be a weak feedback stimulus that unconsciously or subconsciously alerts the wearer, e.g. a feedback stimulus that stops the event but does not wake the wearer. The feedback device may also be provided in the in-ear device, e.g. in the form of a speaker, a vibration motor, or an electrical stimulator.

In embodiments, the processor is further configured to determine when the event terminates, for example by determining when the first and/or second characteristics stop coinciding with the indicative characteristics. The processor may further determine when the event terminates when the first and/or second characteristics drops below the indicative characteristic.

In further embodiments, the feedback device is provided in the first in-ear device for providing the feedback stimulus to the first ear canal, e.g. to provide an audible feedback stimulus to the first ear canal.

In embodiments, an intensity, frequency and/or type of the feedback stimulus is determined by using a reinforcement learning algorithm. Each person may react differently to the biofeedback stimulus. For some wearers a strong biofeedback stimuli is required, while for others the feedback stimuli may be weaker. By allowing reinforcement learning, for example continuous reinforcement learning, the algorithm may deliver and track the effect of varying feedback stimuli on the characteristics of the event and may identify the most suitable timing, intensity, frequency, pattern and/or type of feedback stimuli in reducing e.g., the duration and/or intensity of the event. In this way the feedback stimuli is personalized and dynamic, which prevents adaptation to the feedback stimuli.

In embodiments, the feedback stimulus is one or more of a haptic feedback, an acoustic signal, a mechanical stimulus and an electrical stimulus.

In embodiments, the shape conforming material may be a suitable biocompatible material that is configured to support the magnets or the magnetic sensors in the ear canals and to conform the shape thereof to the shape of the ear canals.

In embodiments, the first in-ear device further comprises a support portion, which, when the first in-ear portion is placed in the first ear channel, extends out of the ear channel, e.g. to support the in-ear device on the ear. The second in-ear device may also comprise a support portion that extends out of the second ear canal. For example, the support portions of the first and second in-ear devices may be connected such that the first and second in-ear devices form a single unit which may be worn in both ear canals.

In embodiments, the first magnetic sensor is provided in the support portion of the first in-ear device. Similarly, the second magnetic sensor may be provided in the support portion of the second in-ear device.

The invention is further related to a method for tracking jaw movement wherein use is made of a jaw movement tracking system according to the invention.

In embodiments of the method, the method comprises:
placing the first in-ear portion in the first ear canal;
measuring the first movement of the first magnet relative to the first magnetic sensor;
determining the first jaw movement based on the measured movement;
determining the one or more first characteristics of the first jaw movement;
comparing the determined first characteristics to the one or more indicative characteristics;
determining of an occurrence of an event based on the comparing of the determined first characteristics to the one or more indicative characteristics; and
outputting the event signal.

In further embodiments of the method, wherein use is made of a jaw movement tracking system wherein the processor is further configured to:
compare the first characteristics to the second characteristics, e.g. by comparing which of the determined characteristics is closer to the one or more indicative characteristics; and
determine a relative relevance of the determined characteristics,
   the method comprises:
placing the first in-ear portion in the first ear canal and the second in-ear portion in the second ear canal;
measuring the first movement of the first magnet relative to the first magnetic sensor and measuring the second movement of the second magnet relative to the second magnetic sensor;
determining the first jaw movement based on the measured first movement and determining the second jaw movement based on the measured second movement;
determining the one or more first characteristics of the first jaw movement and determining the one or more second characteristics of the second jaw movement;
comparing the first characteristics to the second characteristics;
determine a relative relevance of the determined first and second characteristics;
determining of an occurrence of an event based on the most relevant of the first characteristics and the second characteristics;
outputting the event signal.

### BRIEF DESCRIPTION OF THE DRAWINGS

The invention will be explained below with reference to the drawing in which:

FIG. 1 shows a schematic view of a jaw movement tracking system;

FIG. 2 shows a schematic view of an embodiment of the first in-ear device;

FIG. 3 shows a schematic view of another embodiment of the first in-ear device;

FIG. 4 shows an in-ear portion from various sides;

FIG. 5 shows a close-up of the first distal portion of the first in-ear device;

FIG. 6 shows a cross-section of an example of the first distal portion; and

FIG. 7 shows a graph showing a change in flux density over time.

### DESCRIPTION OF THE EMBODIMENTS

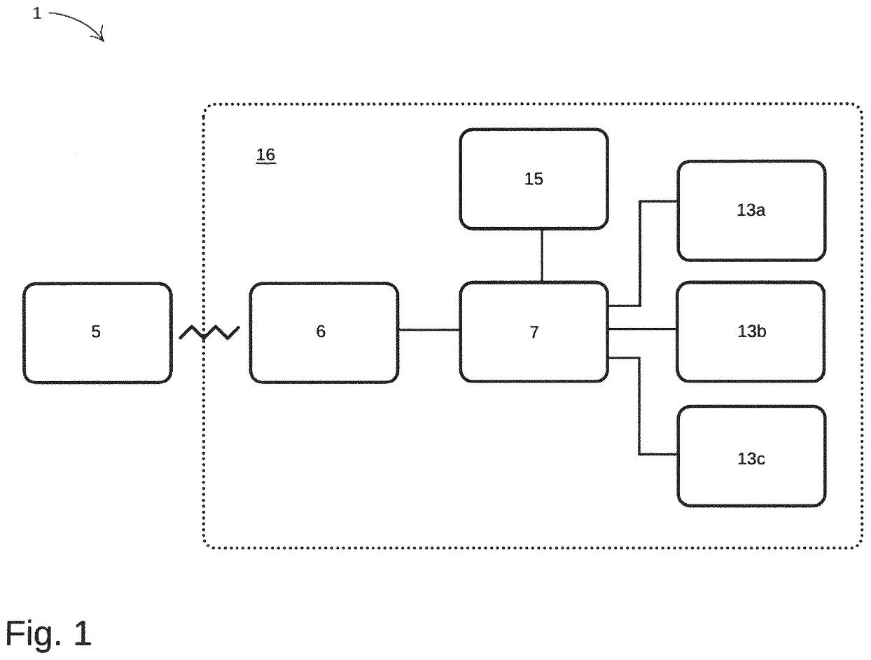

FIG. 1 shows a schematic view of a jaw movement tracking system 1. The tracking system 1 comprises a first in-ear device 2 comprising a first magnet 5 and a first magnetic sensor 6 for measuring a movement of the first magnet 5 relative to the first magnetic sensor 6 by detecting a change in magnetic flux density generated by the relative movement. The magnetic sensor 6 is connected to a processor 7 for receiving the measured relative movement. The processor may be powered by a battery 15.

Either the first magnet 5 or the first magnetic sensor 6 may be provided in the distal portion 4, e.g. wherein the first magnet 5 or the first magnetic sensor 6 is provided adjacent to the temporomandibular joint when the first in-ear portion 3 is placed in the first ear canal. Thus under movement of the first TMJ either the first magnet 5 or the first magnetic sensor 6 may be displaced causing a relative movement of the first magnet 5 relative to the first magnetic sensor 6.

The processor 7 is configured to determine a first jaw movement, e.g. a jaw movement determined by the first in ear device 2, based on the movement of the magnet 5 relative to the magnetic sensor 6. For example, the first jaw movement may be determined based on a computer model, which may be based on an empirical model or a simulation model, which computer model relates the measured relative movement to the first jaw movement. The processor 7 is further configured to determine one or more first characteristics, e.g. characteristics determined based on measurements from the first in-ear device 2. The first characteristics may be velocity, direction, amplitude, and frequency of the first jaw movement. By comparing the determined first characteristics to one or more indicative characteristics that are indicative of a predetermined event, such as bruxism, eating, or TMJ disorders, the occurrence of the event is determined. For example the occurrence of the event is determined when the determined first characteristics overlap, e.g. sufficiently overlap, with the indicative characteristics.

Once the processor 7 determines the occurrence of the event, an output signal is generated and, in the case of FIG. 1, the output signal is sent to one or more feedback devices 13a, b, c, e.g. which feedback devices 13a, b, c are a speaker, vibrator, or a wireless communication device.

In the example of FIG. 1, the first magnet 5 is provided in the distal portion 4 and the processor 7, first magnetic sensor 6, battery 15, and feedback devices 13a, b, c are provided in the proximal portion 15.

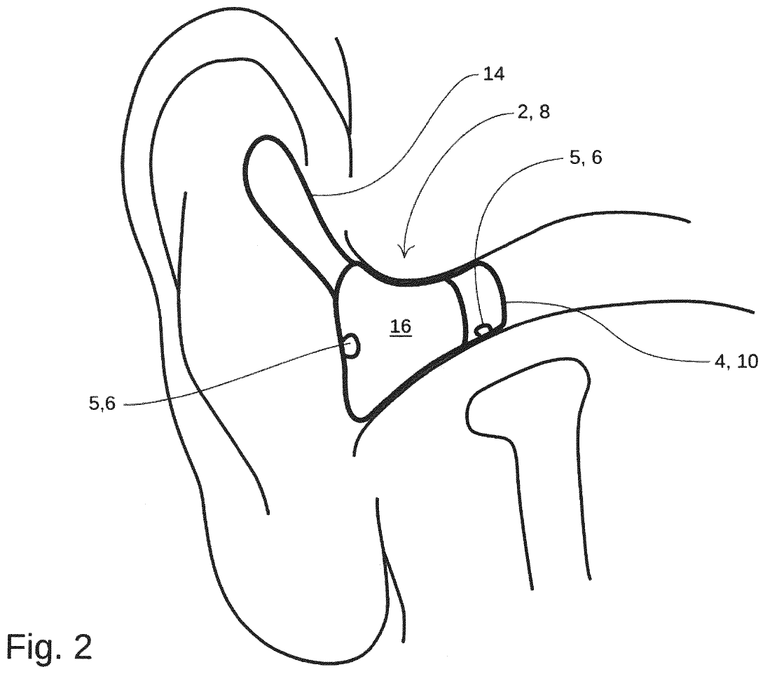

FIG. 2 shows a schematic view of an embodiment of the first in-ear device 2 placed in a first ear canal. The first in-ear device 2 of this embodiment comprises an in-ear portion 3 and a distal portion 4, wherein the distal portion 4 is made of a shape conforming material. The distal portion 4 comprises either the first magnet 5 or the first magnetic sensor 6 provided adjacent the TMJ and the in-ear portion 3 further comprises, opposite the distal portion 3, the other of the first magnet 5 and the first magnetic sensor 6, such that a relative movement of the magnet 5 relative to the first magnetic sensor 6 is measurable by the first magnetic sensor 6. The first in-ear device 2 further comprises a proximal portion 14 which is configured to support the first in-ear device in the first ear canal.

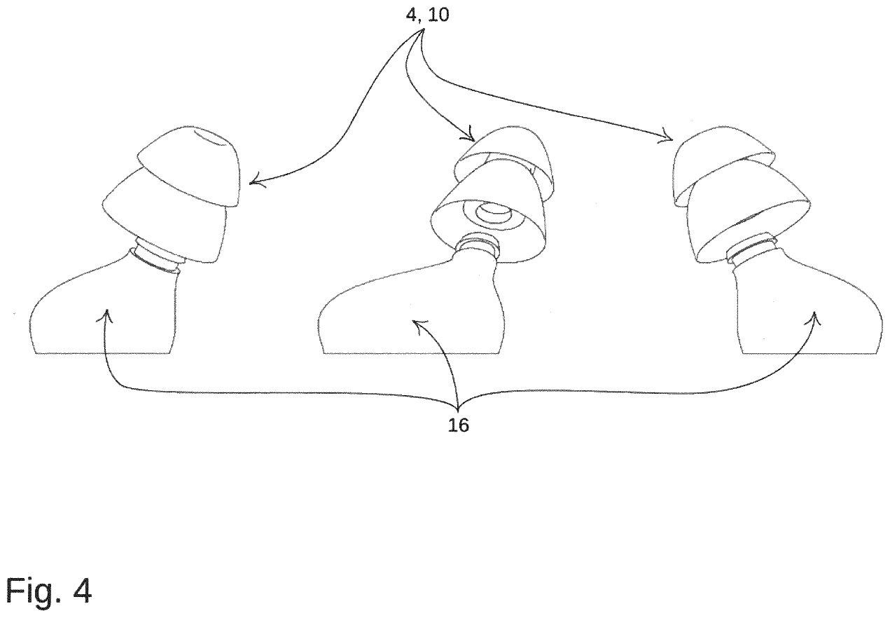

FIG. 4 shows an in-ear portion 3 from various sides showing the first distal portion 4 and the proximal portion 16. As before, the first magnet 5 or the first magnetic sensor 6 is provided in the first distal portion 4 which is made from a shape conforming material and the other one of the first magnet 5 and the first magnetic sensor 6 is placed in the first proximal portion 16. The shape of the in-ear portion may depend on whether or not the first in-ear portion 3 is designed to be placed in a left ear canal or a right ear canal.

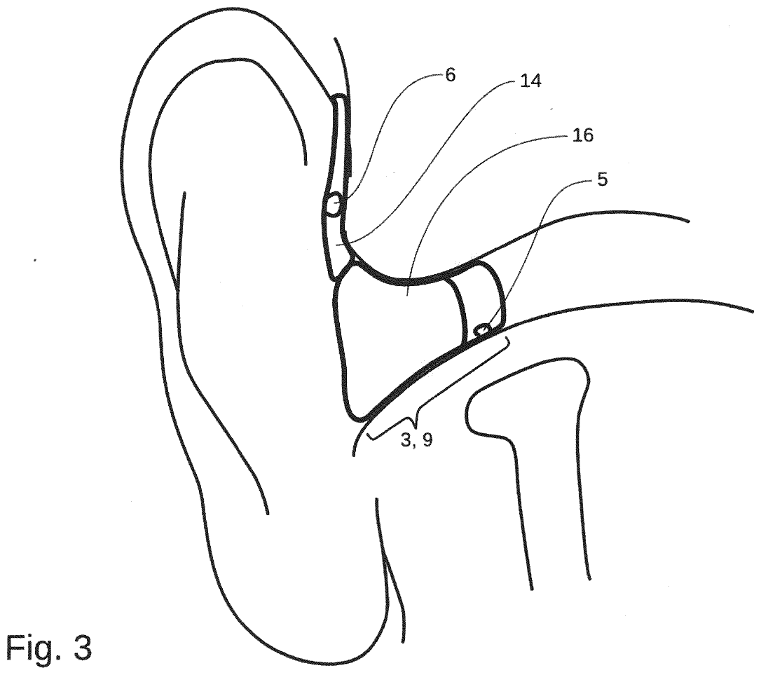

FIG. 3 shows a schematic view of another embodiment of the first in-ear device 3. In this embodiment the first magnet 5 is provided in the first distal portion 4 and the first magnetic sensor 6 is provided in the support portion 14 which supports the in-ear device 3 in the ear canal.

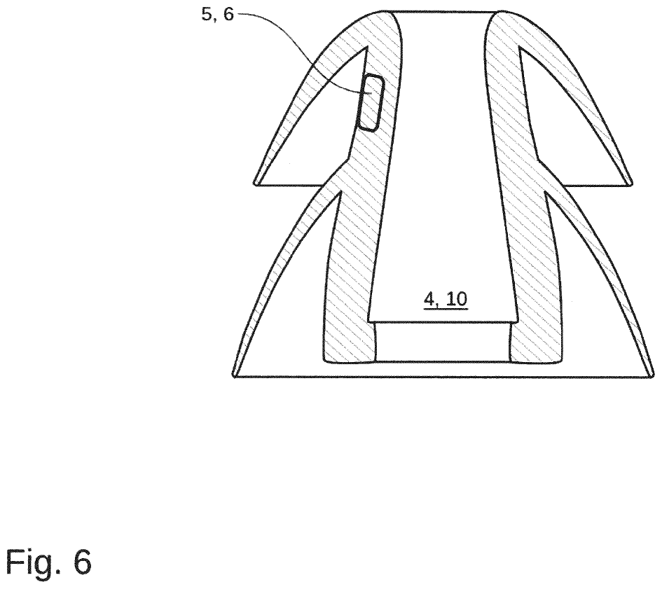

FIG. 6 shows a cross-section of an example of the first distal portion.

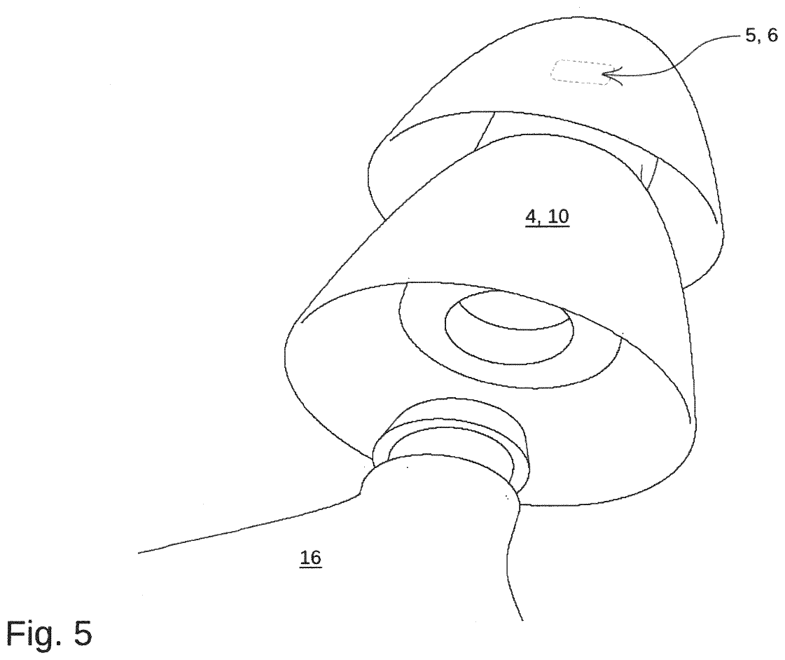

FIG. 5 shows a close-up of the distal portion 4 of the first in-ear device 2. As can be seen in the figure, the first distal portion 4 may be removable from the rest of the first in-ear portion 3, e.g. from the first proximal portion 16. For example, this allows the first distal portion 4, which is in most contact with the first ear canal, to be more easily cleaned or replaced. The first magnet 5 or first magnetic sensor 6 is provided in the first distal portion 4.

As shown in FIG. 6, which shows a cross-section of a first distal portion 4, the first distal portion 4 comprises a first magnet 5. The first magnet 5 may be embodied as a strip running over a portion of the inner surface of the first distal portion 4, which allows the first magnetic sensor 6 to measure relative movement of the first magnet 5 due to changes in the shape of the first ear canal.

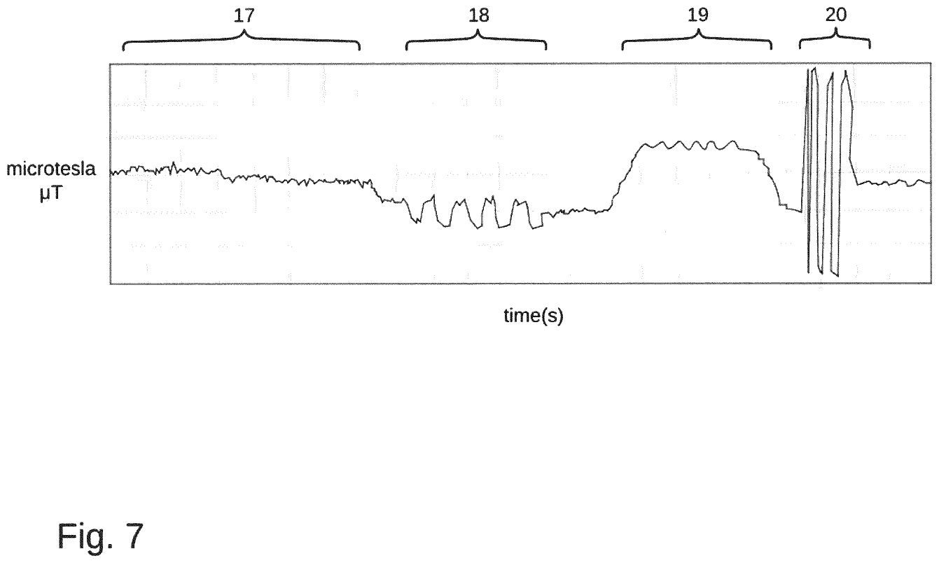

FIG. 7 shows a graph showing a change in flux density over time, wherein different events are indicated. The figure shows a change in flux density over time for a single axis, e.g. an x-axis. As can be seen from the graph, different jaw movements lead to different measured flux density which can then be related to the occurrence of events by the processor 7. For example, the first part 17 of the graph is related to a relaxed jaw, the second part 18 of the graph is related to grinding or clenching of the jaw, the third part 19 of the graph is related to yawning, and the fourth part 20 is related to speaking. As can be seen, different jaw movements lead to distinctive signals in measured flux density.
1 jaw movement tracking system
2 first in-ear device
3 first in-ear portion
4 first distal portion
5 first magnet
6 first magnetic sensor
7 processor
8 second in-ear device
9 second in-ear portion
10 second distal portion
11 second magnet
12 second magnetic sensor
13 feedback device
14 support portion
15 battery
16 first proximal portion

1. A jaw movement tracking system, comprising:
a first in-ear device comprising:
a first in-ear portion configured to be placed in a first ear canal, wherein the first in-ear portion comprises a first distal portion made from a shape conforming material that conforms to a shape of the first ear canal when the first in-ear portion is placed in the first ear canal;
a first magnet;
a first magnetic sensor for measuring movement of the first magnet relative to the first magnetic sensor by detecting a change in magnetic flux density generated by the relative movement; and
a processor connected to the first magnetic sensor for receiving the measured movement;
wherein either the first magnet or the first magnetic sensor is provided in the distal portion,
wherein the processor is configured to:
determine a first jaw movement based on measured relative movement of the first magnet relative to the first magnetic sensor, relating the relative movement to jaw movement;
determine one or more first characteristics of the first jaw movement, based on the determined first jaw movement;
compare the determined first characteristics to one or more indicative characteristics that are indicative of a predetermined event to determine an occurrence of the event, e.g. wherein an occurrence of an event is determined when the determined first characteristics have a predetermined overlap with the indicative characteristics; and
output an event signal when the processor determines the occurrence of the event.

2. The jaw movement tracking system according to claim 1, wherein the in-ear device further comprises:
a second in-ear device comprising:
a second in-ear portion configured to be placed in a second ear canal, wherein the second in-ear portion comprises a second distal portion made from a shape conforming material that conforms to a shape of the second ear canal when the second in-ear portion is placed in the second ear canal;
a second magnet;
a second magnetic sensor for measuring movement of the second magnet relative to the second magnetic sensor by detecting a change in magnetic flux density generated by the relative movement,
wherein either the second magnet is provided in the second distal portion, or wherein the magnetic sensor is provided in the distal portion,
wherein the processor is connected to the second magnetic sensor for receiving the second measured relative movement,
and wherein the processor is further configured to:
determine a second jaw movement based on second measured movement of the second magnet relative to the second magnetic sensor;
determine one or more second characteristics of the second jaw movement, based on the second jaw movement;
compare the determined second characteristics to one or more indicative characteristics that are indicative of a predetermined event to determine an occurrence of the event; and
output the event signal when the processor determines the occurrence of the event.

3. The jaw movement tracking system according to claim 2, wherein the processor is further configured to:
compare the first characteristics to the second characteristics; and
determine a relative relevance of the determined characteristics.

4. The jaw movement tracking system according to claim 1, wherein the predetermined event is one of bruxism, sleep stage, sleep-breathing episode, eating, abnormal temporomandibular joint movement, provide a biometric authentication via silent speech recognition, jaw movement for providing hands-free jaw gesture control for a device, or smoking.

5. The jaw movement tracking system according to claim 1, wherein the first magnetic sensor and/or the second magnetic sensor is a hall effect sensor.

6. The jaw movement tracking system according to claim 1, wherein the first magnetic sensor and/or the second magnetic sensor are configured to determine a relative rotational movement of the first and/or second magnet, and/or a relative linear movement of the first and/or second magnet.

7. The jaw movement tracking system according to claim 1, wherein the tracking system further comprises a feedback device connected to the processor, wherein the feedback device is configured to provide a feedback stimulus when the feedback device receives the event signal from the processor.

8. The jaw movement tracking system according to claim 7, wherein the feedback device is provided in the first in-ear device for providing the feedback stimulus to the first ear canal.

9. The jaw movement tracking system according to claim 7, wherein an intensity, frequency and/or type of the feedback stimulus is determined by using a reinforcement learning algorithm.

10. The jaw movement tracking system according to claim 7, wherein the feedback stimulus is one or more of a haptic feedback, an acoustic signal, a mechanical stimulus and or an electrical stimulus.

11. The jaw movement tracking system according to claim 1, wherein the shape conforming material is a suitable biocompatible material that is configured to support the magnets or the magnetic sensors in the ear canals and to conform the shape thereof to the shape of the ear canal.

12. The jaw movement tracking system according to claim 1, wherein the first in-ear device further comprises a support portion which, when the first in-ear portion is placed in the first ear canal extends out of the ear canal.

13. The jaw movement tracking system according to claim 12, wherein the first magnetic sensor is provided in the support portion of the first in-ear device.

14. A method for tracking jaw movement wherein use is made of a jaw movement tracking system according to claim 1.

15. The method according to claim 14, wherein the method comprises:
placing the first in-ear portion in the first ear canal;
measuring the first movement of the first magnet relative to the first magnetic sensor;
determining the first jaw movement based on the measured movement;
determining the one or more first characteristics of the first jaw movement;
comparing the determined first characteristics to the one or more indicative characteristics;
determining of an occurrence of an event based on the comparing of the determined first characteristics to the one or more indicative characteristics; and
outputting the event signal.

16. The method according to claim 14, wherein use is made of a jaw movement tracking system at least according to claim 3, wherein the method comprises:
placing the first in-ear portion in the first ear canal and the second in-ear portion in the second ear canal;
measuring the first movement of the first magnet relative to the first magnetic sensor and measuring the second movement of the second magnet relative to the second magnetic sensor;
determining the first jaw movement based on the measured first movement and determining the second jaw movement based on the measured second movement;
determining the one or more first characteristics of the first jaw movement and determining the one or more second characteristics of the second jaw movement;
comparing the first characteristics to the second characteristics;
determine a relative relevance of the determined first and second characteristics;
determining of an occurrence of an event based on the most relevant of the first characteristics and the second characteristics; and
outputting the event signal.
